(19) United States
(12) Patent Application Publication
(10) Pub. No.: US 2010/0221322 A1
(43) Pub. Date: Sep. 2, 2010

(54) Abuse-Resistant Pharmaceutical Dosage Form

(75) Inventors: Johannes Bartholomäus, Aachen (DE); Klaus-Dieter Langner, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/778,521

(22) Filed: May 12, 2010

Related U.S. Application Data
(63) Continuation of application No. 11/113,020, filed on Apr. 25, 2005, now abandoned.
Application No. 11/113,020 is a continuation of application No. PCT/EP2003/011785, filed on Oct. 24, 2003.

(30) Foreign Application Priority Data
Oct. 25, 2002 (DE) DE 102 50 088.6

(51) Int. Cl. A61K 9/48; A61K 31/485; A61K 9/30; A61K 9/36; A61P 25/04

(52) U.S. Cl. 424/451; 514/282; 424/474; 424/480

(57) ABSTRACT

A solid pharmaceutical dosage form that is safeguarded against abuse containing at least one active substance that could be subject to abuse and at least one antagonist for the active substance, which antagonist is spatially separate from the active substance. The active substance or substances is/are present in at least one subunit (a), and the at least one antagonist is present in at least one subunit (b), and the at least one antagonist in subunit (b) is to all intents and purposes not released in the body if the dosage form is correctly administered as prescribed.

### CROSS REFERENCE TO RELATED APPLICATIONS

This application is a continuation of co-pending U.S. application Ser. No. 11/113,020, filed Apr. 25, 2005, which in turn was a continuation of international patent application no. PCT/EP2003/011785, filed Oct. 24, 2003, designating the United States of America, and published in German on May 6, 2004 as WO 2004/037260, the entire disclosures of which are incorporated herein by reference. Priority is claimed based on Federal Republic of Germany patent application no. DE 102 50 088.6, filed Oct. 25, 2002.

### BACKGROUND OF THE INVENTION

The present invention relates to an abuse-proofed, solid dosage form comprising at least one active ingredient with potential for abuse and at least one antagonist for this active ingredient spatially separate therefrom, wherein the active ingredient or active ingredients is/are present in at least one subunit (a) and the antagonist or antagonists are present in at least one subunit (b) and, in the event of correct administration of the dosage form, the antagonist or antagonists is/are practically not released in the body from subunit (b).

Many pharmaceutical active ingredients, in addition to having excellent activity in their appropriate application, also have potential for abuse, i.e. they can be used by an abuser to bring about effects other than the medical ones intended. Opiates, for example, which are highly active in combating severe to very severe pain, are frequently used by abusers to achieve a state of narcosis or euphoria.

Oral dosage forms which contain such active ingredients with potential for abuse do not usually give rise to the result desired by the abuser, even when taken in an abusively large quantity, because blood levels of the active ingredients increase only slowly. In order nevertheless to enable abuse, the corresponding dosage forms are comminuted, for example ground, by the abuser and administered, for example, by sniffing nasally. In another form of abuse, the active ingredient is extracted from the powder obtained by comminution of the dosage form using a preferably aqueous liquid and the resultant solution, optionally after being filtered through cotton wool or cellulose wadding, is administered parenterally, in particular intravenously. These forms of administration give rise to an accelerated rise in levels of the active ingredient, relative to oral administration, providing the abuser with the desired result.

In order to prevent this form of the abuse, U.S. Pat. No. 5,866,164 (=WO 97/33566) proposes a dosage form in the form of an oral osmotic therapeutic system with a two-layer core, wherein the first layer of the core, facing towards the opening of the system comprises an opioid analgesic and the second layer comprises an antagonist for this opioid analgesic and simultaneously effects the push function, i.e. expelling the analgesic from the corresponding layer out of the opening of the system.

U.S. Pat. No. 6,277,384 discloses a dosage form containing a combination of an opioid agonist and an opioid antagonist in a specific ratio, which brings about a negative effect on administration to an addicted person.

U.S. Pat. No. 6,228,863 describes a dosage form containing a combination of an opioid agonist and an opioid antagonist, the formulation of which has been selected such that the two compounds can in each case only be extracted together from the dosage form and then an at least two-stage process is required to separate them.

All these dosage forms described in the prior art have the disadvantage that, even when they are correctly administered, the corresponding antagonist is in each case also released in addition to the opioid active ingredient. This means that the activity of the opioid agonist is impaired and it is necessary to increase the quantity thereof required in the dosage form for satisfactory treatment of the patient. The risk of the occurrence of undesirable accompanying symptoms is increased in comparison with dosage forms which contain no opioid antagonists.

Moreover it is fundamentally desirable when such a dosage form is correctly administered not to increase still further the stress on the patient by the released proportion of opioid antagonist.

### SUMMARY OF THE INVENTION

The object of the present invention was accordingly to provide a dosage form which is not subject to the aforementioned disadvantages of the prior art.

This object has been achieved by the abuse-resistant, solid dosage form according to the invention which comprises at least one active ingredient with potential for abuse and at least one antagonist for this active ingredient spatially separate therefrom, wherein the active ingredient or active ingredients is/are present in at least one subunit (a) and the antagonist or antagonists are present in at least one subunit (b) and, in the event of correct administration of the dosage form, the antagonist or antagonists is/are practically not released in the body from subunit (b).

For the purposes of the present invention, subunits are solid formulations which in each case comprise only the active ingredient(s) or only the antagonist(s) in addition to conventional auxiliary substances known to the person skilled in the art. Methods for producing corresponding subunits are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding description is hereby incorporated by reference and is deemed to be part of the disclosure.

The dosage form according to the invention may comprise in each of its respective subunits (a) and (b) one or more active ingredients with potential for abuse and one or more antagonists. The dosage form according to the invention preferably comprises in the corresponding subunits in each case only one active ingredient and only one antagonist for this active ingredient.

Pharmaceutical active ingredients with potential for abuse are known to those skilled in the art, as are the quantities thereof to be used and processes for the production thereof, and may be present in the dosage form according to the invention as such, in the form of corresponding derivatives, in particular esters or ethers, or in each case in the form of corresponding physiologically acceptable compounds, in particular in the form of the salts or solvates thereof.

The dosage form according to the invention is particularly suitable for preventing abuse of a pharmaceutical active ingredient selected from the group consisting of opiates, opioids, stimulants and further narcotics.

The dosage form according to the invention is especially suitable for preventing abuse of opiates, opioids and further narcotics which are selected from the group consisting of N-{1-[2-(4-ethyl-5-oxo-2-tetrazolin-1-yl)ethyl]-4-methoxymethyl-4-piperidyl}propionanilide (alfentanil), allylprodine, alphaprodine, 2-diethylaminopropiophenone (amfepramone), (±)-α-methylphenethylamine (amphetamine), 2-(α-methylphenethylamino)-2-phenylacetonitrile (amphetaminil), anileridine, apocodeine, benzylmorphine, bezitramide, 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), butorphanol, (1S,2S)-2-amino-1-phenyl-1-propanol (cathine/D-norpseudoephedrine), clonitazene, (−)-methyl-[3β-benzoyloxy-2β(1αH,5αH)-tropane carboxylate] (cocaine), 4,5α-epoxy-3-methoxy-17-methyl-7-morphinen-6α-ol (codeine), cyclorphan, cyprenorphine, desomorphine, dextromoramide, (+)-(1-benzyl-3-dimethylamino-2-methyl-1-phenylpropyl) propionate (dextropropoxyphene), dezocine, diampromide, diamorphone, 4,5α-epoxy-3-methoxy-17-methyl-6α-morphinanol (dihydrocodeine), 4,5α-epoxy-17-methyl-3,6α-morphinandiol (dihydromorphine) dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, ethoheptazine, ethylmethylthiambutene, 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-ethenol-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-(α-methylphenethylamino)propionitrile (fenproporex), N-(1-phenethyl-4-piperidyl)propionanihde (fentanyl), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LRAM)), (−)-6-dimethylamino-4,4-diphenol-3-heptanone (levomethadone), (−)-17-methyl-3-morphinanol (levorphanol), levophenacylmorphane, lofentanil, 5-(4-chlorophenyl)-2,5-dihydro-3H-imidazo[2,1-α]isoindol-5-ol (mazindol), N-(3-chloropropyl)-α-methylphenethylamine (mefenorex), meperidine, meptazinol, metazocine, methylmorphine, N,α-dimethylphenethylamine (metamphetamine), (±)-6-dimethylamino-4,4-diphenol-3-heptanone (methadone), methyl[2-phenyl-2-(2-piperidyl)acetate](methylphenidate), 3,3-diethyl-5-methyl-2,4-piperidinedione (methyprylon), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10, α-tetrahydro-1-hydroxy-6,6-dimethyl-6H-dibenzo-[b,d]pyran-9(6αH)-one (nabilone), nalbuphene, narceine, nicomorphine, norlevorphanol, 6-dimethylamino-4,4-diphenyl-3-hexanone (normethadone), normorphine, norpipanone, the exudation from plants belonging to the species Papaver somniferum (opium), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papavereturn, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), dimethylphenethylamine (phentermine), α-(2-piperidyl)benzhydryl alcohol (pipradrol), 1′-(3-cyano-3,3-diphenylpropyl)[1,4′-bipiperidine]-4′-carboxamide (piritramide), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate}(remifentanil), N-{4-methoxymethyl-1-[2-(2-thienyl)ethyl]-4-piperidyl}propionanilide (sufentanil), ethyl (2-dimethylamino-1-phenyl-3-cyclohexene-1-carboxylate) (tilidine, cis and trans)), tramadol, (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, in each case optionally in the form of corresponding stereoisomeric compounds and corresponding derivatives, in particular esters or ethers, and in each case physiologically acceptable compounds, in particular salts and solvates.

The compounds (1R*,2R*)-3-(3-dimethylamino-1-ethyl-2-methyl-propyl)-phenol and (1R,2R,4S)-2-(dimethylamino)methyl-4-(p-fluorobenzyloxy)-1-(m-methoxyphenyl)cyclohexanol, the physiologically acceptable compounds thereof, in particular the hydrochlorides thereof and processes for the production thereof are respectively known, for example, from U.S. Pat. No. 6,248,737 (=EP 693,475) and U.S. Pat. No. 5,801,201 (=EP 780,369), the disclosures of which are hereby incorporated by reference.

The dosage form according to the invention is also suitable for preventing abuse of stimulants, preferably those selected from the group consisting of amphetamine, norpseudoephedrine, methylphenidate and their respective corresponding physiologically acceptable compounds thereof, in particular the bases, salts and solvates thereof.

Suitable antagonists for the particular active ingredients for preventing the abuse thereof are known per se to those skilled in the art and may be present in the dosage forms according to the invention as such or in the form of corresponding derivatives, in particular esters or ethers, or in each case in the form of corresponding physiologically acceptable compounds, in particular in the form of the salts or solvates thereof in conventional quantities known to the person skilled in the art.

If subunit (a) comprises an opiate or an opioid as active ingredient, the antagonist used in subunit (b) is preferably naloxone, naltrexone, nalmefene, nalide, nalmexone, nalorphine or naluphine, in each case optionally in the form of a corresponding physiologically acceptable compound, in particular in form of a base, a salt or solvate. The dosage form according to the invention preferably comprises the corresponding antagonists in a quantity of ≧10 mg, particularly preferably in a quantity of 10 to 100 mg, very particularly preferably in a quantity of 10 to 50 mg per dosage form, i.e. per administration unit.

If the dosage form according to the invention comprises a stimulant as the active ingredient, the antagonist is preferably a neuroleptic, preferably selected from the group consisting of haloperidol, promethazine, fluphenazine, perphenazine, levomepromazine, thioridazine, perazine, chlorpromazine, chlorprothixine, zuclopentixol, flupentixol, prothipendyl, zotepine, benperidol, pipamperone, melperone, bromperidol. The dosage form according to the invention preferably comprises the corresponding antagonists in a conventional therapeutic dose known to the person skilled in the art, particularly preferably in a quantity of twice to four times the conventional dose per administration unit.

A substantial aspect of the present invention is that the antagonist is not released or is hardly released (i.e., is not released in an effective amount) in the body by the subunit or subunits (b) of the dosage form according to the invention when administered correctly. Persons skilled in the art will understand that the formulation conditions necessary for this purpose may vary as a function of the antagonist used in each case and the formulation of subunit (b) or the dosage form. The optimum formulation for the particular antagonist may be determined by simple preliminary testing.

If the dosage form according to the invention is manipulated for the purpose of abusive taking of the active ingredient, e.g. by grinding and optionally extracting the powder thus obtained with a suitable extracting agent, in addition to the active ingredient, the antagonist is also obtained in a form in which it cannot easily be separated from the active ingredient, such that, on administration of the manipulated dosage form, in particular in the case of nasal and/or parenteral administration, the active ingredient cannot exert the effect desired by the abuser.

The dosage form according to the invention may be formulated in a large number of ways according to conventional methods known to the person skilled in the art, wherein the subunits (a) and (b) in the dosage form according to the invention may each be present in any spatial arrangement relative to one another, provided that it is ensured that the antagonist is practically not released in the body. Methods for formulating the dosage forms are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding description is hereby incorporated by reference and is deemed to be part of the disclosure.

In one preferred embodiment of the dosage form according to the invention, both subunits (a) and (b) are present in multiparticulate form, wherein microtablets, microcapsules, micropellets, granules, spheroids, beads or pellets are preferred and the same form, i.e. shape, is selected for both subunit (a) and subunit (b), such that it is not possible to separate subunits (a) from (b) by mechanical selection. The multiparticulate forms are preferably of a size in the range from 0.1 to 3 mm, particularly preferably from 0.5 to 2 mm in size.

The subunits (a) and (b) in multiparticulate form may also preferably be packaged in a capsule, suspended in a liquid or a gel or be press-molded to form a tablet, wherein the final formulation in each case proceeds in such a manner that the subunits (a) and (b) are also retained in the resultant dosage form.

The respective multiparticulate subunits (a) and (b) of identical shape must also not be visually distinguishable from one another so that the abuser cannot separate them from one another by simple sorting. This may, for example, be achieved by the application of identical coatings which, apart from this disguising function, may also incorporate further functions, such as, for example, delayed release of one or more active ingredients or provision of a finish resistant to gastric juices on the particular subunits.

In a further preferred embodiment of the present invention, the respective subunits (a) and (b) are arranged in layers relative to one another. The layered subunits (a) and (b) are preferably arranged for this purpose vertically or horizontally relative to one another in the dosage form produced according to the invention, wherein in each case one or more layered subunits (a) and one or more layered subunits (b) may be present in the dosage form, such that, apart from the preferred layer sequences (a)-(b) or (a)-(b)-(a), any desired other layer sequences may be considered.

Another preferred dosage form according to the invention is one in which subunit (b) forms a core which is completely enclosed by subunit (a). Such a structure is preferably also suitable for the above-stated multiparticulate forms, wherein both subunits (a) and (b) and an optionally present separation layer (c) therebetween are then formulated in one and the same multiparticulate form.

In a further preferred embodiment of the dosage form according to the invention, the subunit (a) forms a core, which is enclosed by subunit (b), wherein subunit (b) further comprises at least one channel which leads from the core to the surface of the dosage form.

The dosage form according to the invention may comprise, between one layer of the subunit (a) and one layer of the subunit (b), in each case one or more, preferably one, optionally swellable separation layer (c) which serves to separate subunit (a) spatially from (b). If the dosage form according to the invention comprises the layered subunits (a) and (b) and an optionally present separation layer (c) in an at least partially vertical or horizontal arrangement, the dosage form preferably assumes the form of a tablet, a coextrudate or a laminate.

In one particularly preferred embodiment, the entirety of the free surface of subunit (b) and optionally at least part of the free surface of subunit(s) (a) and optionally at least part of the free surface of the optionally present separation layer(s) (c) may be coated with at least one barrier layer (d) which prevents release of the antagonist.

Another particularly preferred embodiment of the dosage form according to the invention comprises a vertical or horizontal arrangement of the layers of subunits (a) and (b) and at least one push layer (p) arranged therebetween, and optionally a separation layer (c), in which dosage form the entirety of the free surfaces of the layer structure consisting of subunits (a) and (b), the push layer and the optionally present separation layer (c) are provided with a semipermeable coating (e), which is permeable to a release medium, i.e. conventionally a physiological liquid, but substantially impermeable to the active ingredient and to the antagonist, and wherein this coating (e) comprises at least one opening for release of the active ingredient in the area of subunit (a).

A corresponding dosage form is known to the person skilled in the art, for example under the name oral osmotic therapeutic system (OROS), as are suitable materials and methods for the production thereof, inter alia from U.S. Pat. Nos. 4,612,008; 4,765,989 and 4,783,337, the disclosures of which are incorporated herein by reference. If the dosage form according to the invention assumes the form of an OROS, subunits (a) and (b) are spatially separated from one another, preferably by a swellable separation layer which is simultaneously the push layer of the OROS.

In a further preferred embodiment, the subunit (a) of the dosage form according to the invention assumes the form of a tablet having a peripheral edge face and two main faces, wherein the edge face and optionally one of the two main faces is covered with a barrier layer (d) containing the antagonist.

Those skilled in the art will understand that the auxiliary substances of the subunit(s) (a) or (b) and of the optionally present separation layer(s) (c) and/or of the barrier layer(s) (d) used in formulating the dosage form according to the invention will vary depending on the arrangement thereof in the dosage form according to the invention, the mode of administration and on the particular active ingredient and the antagonist used. Materials which have the requisite properties are well known to those skilled in the art.

If release of the antagonist from subunit (b) of the dosage form according to the invention is prevented with the assistance of a cover, preferably a barrier layer, the subunit may consist of conventional materials known to the person skilled in the art.

If a corresponding barrier layer (d) is not provided to prevent release of the antagonist, the materials of the subunits should be selected such that release of the antagonist from subunit (b) is virtually ruled out. The materials which are stated below to be suitable for production of the barrier layer may preferably be used for this purpose:

Preferred materials include those which are selected from the group consisting of alkylcelluloses hydroxyalkylcelluloses, glucans, scleroglucans, mannans, xanthans, copolymers of poly[bis(p-carboxyphenoxy)propane and sebacic acid], preferably in a molar ratio of 20:80 (marketed under the name Polifeprosan 20®), carboxymethylcelluloses, cellulose ethers, cellulose esters, nitrocelluloses, polymers based on (meth)acrylic acid and the esters thereof, polyamides, polycarbonates, polyalkylenes, polyalkylene glycols, polyalkylene oxides, polyalkylene terephthalates, polyvinyl alcohols, polyvinyl ethers, polyvinyl esters, halogenated polyvinyls, polyglycolides, polysiloxanes and polyurethanes and the copolymers thereof.

Particularly suitable materials may be selected from the group consisting of methylcellulose, ethylcellulose, hydroxypropylcellulose, hydroxypropylmethylcellulose, hydroxybutylmethylcellulose, cellulose acetate, cellulose propionate (of low, medium or high molecular weight), cellulose acetate propionate, cellulose acetate butyrate, cellulose acetate phthalate, carboxymethylcellulose, cellulose triacetate, sodium cellulose sulfate, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctatdecyl acrylate, polyethylene, low density polyethylene, high density polyethylene, polypropylene, polyethylene glycol, polyethylene oxide, polyethylene terephthalate, polyvinyl alcohol, polyvinyl isobutyl ether, polyvinyl acetate and polyvinyl chloride.

Especially suitable copolymers may be selected from the group consisting of copolymers of butyl methacrylate and isobutyl methacrylate, copolymers of methyl vinyl ether and maleic acid of high molecular weight, copolymers of methyl vinyl ether and maleic acid monoethyl ester, copolymers of methyl vinyl ether and maleic anhydride and copolymers of vinyl alcohol and vinyl acetate.

Further biodegradable materials which are particularly suitable for formulating the barrier layer include starch-filled polycaprolactone [WO98/20073], aliphatic polyesteramides [U.S. Pat. No. 6,344,535 (=DE 19 753 534); CA 2,317,747 (=DE 19 800 698); U.S. Pat. No. 5,928,739 (=EP 820,698)], aliphatic and aromatic polyester urethanes [U.S. Pat. No. 6,821,588 DE 19822979)], polyhydroxyalkanoates, in particular polyhydroxybutyrates, polyhydroxyvalerates, casein [U.S. Pat. No. 5,681,517 (=DE 4 309 528)], polylactides and copolylactides [U.S. Pat. No. 6,235,825 (=EP 980,894)], the disclosures of which are incorporated herein by reference.

The above-stated materials may optionally be blended with further conventional auxiliary substances known to those skilled in the art, preferably selected from the group consisting of glyceryl monostearate, semi-synthetic triglyceride derivatives, semi-synthetic glycerides, hydrogenated castor oil, glyceryl palmitostearate, glyceryl behenate, polyvinylpyrrolidone, gelatine, magnesium stearate, stearic acid, sodium stearate, talcum, sodium benzoate, boric acid and colloidal silica, fatty acids, substituted triglycerides, glycerides, polyoxyalkylene glycols and the derivatives thereof.

If the dosage form according to the invention comprises a separation layer (c), this separation layer, like the subunit (b) not covered by a barrier layer, may preferably be comprised of the above-stated materials described for the barrier layer. Those skilled in the art will understand that release of the antagonist from the particular subunit may be prevented by the thickness of the separation layer.

The dosage form according to the invention for oral administration of one or more active ingredients is particularly suitable for preventing oral, nasal and/or parenteral abuse of such active ingredients.

One or more active ingredients at least partially in delayed-release form may also be present, wherein delayed release may be achieved with the assistance of conventional materials and methods known to persons skilled in the art, for example by embedding the active ingredient in a delayed-release matrix or by the application of one or more delayed-release coatings. Active ingredient release must, however, be controlled such that, in the event of correct administration of the dosage form, the antagonist is practically not released in the body.

If the dosage form according to the invention is intended for oral administration, it may also preferably comprise a coating which is resistant to gastric juices and dissolves as a function of the pH value of the release environment. By means of this coating, it is possible to ensure that the dosage form according to the invention passes through the stomach undissolved and the active ingredient is only released in the intestines. The coating which is resistant to gastric juices preferably dissolves at a pH value of between 5 and 7.5. In this embodiment too, subunit (b) should of course also be formulated such that the antagonist is practically not released in the body.

Corresponding materials and methods for the delayed release of active ingredients and for the application of coatings which are resistant to gastric juices are described, for example, in “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding description is hereby incorporated by reference and is deemed to be part of the disclosure.

The dosage forms according to the invention have the advantage that they are protected against nasal and/or parenteral abuse, without there being any need to worry about placing unnecessary stress on the patient being treated or reducing the efficacy of the respective active ingredient in the event of correct administration. They may be produced simply and comparatively economically.

The invention is in further detail explained below with reference to illustrative examples. These explanations are given merely by way of example and do not restrict the overall scope of the invention.

### EXAMPLES

The quantities indicated below relate in each case to an individual dosage form. A batch from a single production run comprised 1000 dosage forms.

### Example 1

### Jacketed Tablets

### Core

| | |
|---|---|
| Naltrexone hydrochloride | 50 mg |
| Hydrogenated castor oil (Cutina HR) | 50 mg |

Naltrexone hydrochloride and finely powdered hydrogenated castor oil were mixed and press-molded in a tablet press to form round, biconvex tables of a diameter of 6.5 mm.

### Jacket

| | |
|---|---|
| Morphine sulfate pentahydrate | 60 mg |
| Methylhydroxypropylcellulose 100,000 | 100 mg |
| mPa · s (Metolose 90 SH 100,000, ShinEtsu) | |
| Microcrystalline cellulose (Avicel PH 102) | 165 mg |
| Lactose monohydrate | 165 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the 6.5 mm core was inserted centrally, the remaining 250 mg of jacket mixture were added and the jacket was pressed around the core.

### Example 2

### Jacketed Tablets

### Core

| | |
|---|---|
| Naltrexone hydrochloride | 50 mg |
| Hydrogenated castor oil (Cutina HR) | 50 mg |

Naltrexone hydrochloride and finely powdered hydrogenated castor oil were mixed and press-molded in a tablet press to form round, biconvex tables of a diameter of 6.5 mm.

### Jacket

| | |
|---|---|
| Oxycodone hydrochloride | 30 mg |
| Spray-dried lactose | 300 mg |
| Eudragit RSPM | 70 mg |
| Stearyl alcohol | 115 mg |
| Magnesium stearate | 5 mg |
| Talcum | 10 mg |

Oxycodone hydrochloride, spray-dried lactose and Eudragit RSPM were intimately mixed together for approx. 5 min in a suitable mixer. During mixing, the mixture was granulated with such a quantity of purified water that a moist, granulated mass was formed. The resultant granular product was dried in a fluidised bed at 60° C. and passed through a 2.5 mm screen. The granular product was then dried again as described above and passed through a 1.5 mm screen. The stearyl alcohol was melted at 60-70° C. and added to the granular product in a mixer. After cooling, the mass was pressed together with magnesium stearate and talcum through a 1.5 mm screen. From the resultant granular product, approx. 265 mg of the mixture were placed in the tablet die in a tablet press with a tool for 13 mm biconvex tablets, the 6.5 mm core was inserted centrally, the remaining 265 mg of the jacket mixture were added and the jacket was pressed around the core.

### Example 3

### Jacketed Tablets

### Core

| | |
|---|---|
| Naloxone hydrochloride dehydrate | 20 mg |
| Spray-dried lactose | 76 mg |
| Magnesium stearate | 2 mg |
| Colloidal silicon dioxide | 2 mg |

All the constituents were mixed and press-molded in a tablet press to form round, biconvex tablets of a diameter of 6.5 mm.

### Coating on Core

| | |
|---|---|
| Cellulose acetate with 39.8% acetate | 9.5 mg |
| Macrogol 3350 | 0.5 mg |

The coating constituents were dissolved in an acetone-water mixture (95:5 parts by weight) and sprayed onto the cores.

### Jacket

| | |
|---|---|
| Morphine sulfate pentahydrate | 60 mg |
| Methylhydroxypropylcellulose 100,000 | 100 mg |
| mPa · s (Metolose 90 SH 100,000, ShinEtsu) | |
| Microcrystalline cellulose (Avicel PH 102) | 165 mg |
| Lactose monohydrate | 165 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the core coated with cellulose acetate was inserted centrally, the remaining 250 mg of jacket mixture were added and the jacket was pressed around the core.

### Example 4

### Multiparticulate Form

Antagonist Pellets:

| | |
|---|---|
| Naltrexone hydrochloride | 50 mg |
| Lactose | 15 mg |
| Microcrystalline cellulose PH 101 | 30 mg |
| Low-substituted hydroxypropylcellulose (LH31, Shin-Etsu) | 5 mg |

All the constituents were intimately mixed together for approx. 5 min in a suitable mixer. During mixing, the mixture was granulated with such a quantity of purified water that a moist, granulated mass was formed. The resultant granular product was extruded in a Nica extruder through a die with extrusion orifices of 1 mm, rounded for 5 min in a spheroniser, dried in a fluidised bed at 60° C. and classified by means of a 1.5 mm and a 0.5 mm screen.

### Coating on Antagonist Pellets

| | |
|---|---|
| Cellulose acetate with 39.8% acetate | 9.5 mg |
| Macrogol 3350 | 0.5 mg |
| Titanium dioxide | 0.5 mg |

### Quantities Stated Per 100 mg of Antagonist Pellets

Cellulose acetate and macrogol were dissolved in an acetone-water mixture (95:5 parts by weight), titanium dioxide was dispersed in the mixture and the cores were sprayed with the suspension in a fluidised bed unit until the mass of the coated pellets amounted to 110% of the weight of the introduced uncoated pellets.

### Analgesic Pellets

| | |
|---|---|
| 0.5 mm nonpareils (sucrose-maize starch starter pellets, supplied | 50 mg |
| by Werner) | |
| Morphine sulfate pentahydrate | 60 mg |
| Povidone K30 | 30 mg |
| Talcum | 10 mg |

Morphine sulfate and povidone were dissolved in purified water and talcum was dispersed in the solution. The suspension was sprayed onto the nonpareils at 60° C. and dried. The pellets were classified using a 1.5 mm screen and a 0.5 mm screen.

### Coating on Analgesic Pellets

| | |
|---|---|
| Ethylcellulose dispersion (Aquacoat ECD30, FMC Corporation) | 10.0 mg |
| Glycerol monostearate | 2.0 mg |
| Talcum | 2.0 mg |
| Titanium dioxide | 1.0 mg |

Quantities stated per 150 mg of analgesic pellets, weight of ethylcellulose stated as the dry weight obtained from the 30% dispersion of the commercial product.

The ethylcellulose dispersion was mixed 1:0.5 with purified water and the glycerol monostearate was incorporated by stirring for at least two hours. Talcum and titanium dioxide were dispersed in 0.5 parts of water (calculated on the basis of the 1:0.5 mixture of the ethylcellulose dispersion) and mixed with the ethylcellulose dispersion. The analgesic pellets were sprayed with the dispersion in a fluidised bed unit until the mass of the coated pellets amounted to 110% of the weight of the introduced uncoated pellets.

### Final Formulation in Capsules

110 mg of coated pellets containing antagonist and 165 mg of coated analgesic pellets per capsule were mixed and packaged in size 1 hard gelatine capsules.

### Example 5

### Multiparticulate Form

Antagonist pellets:

| | |
|---|---|
| Naloxone hydrochloride dihydrate | 20 mg |
| Lactose | 7 mg |
| Microcrystalline cellulose PH 101 | 20 mg |
| Low-substituted hydroxypropylcellulose (LH31, Shin-Etsu) | 3 mg |

All the constituents were intimately mixed together for approx. 5 min in a suitable mixer. During mixing, the mixture was granulated with such a quantity of purified water that a moist, granulated mass was formed. The resultant granular product was extruded in a Nica extruder through a die with extrusion orifices of 1 mm, rounded for 5 min in a spheroniser, dried in a fluidised bed at 60° C. and classified by means of a 1.5 mm and a 0.5 mm screen.

### Coating on Antagonist Pellets

| | |
|---|---|
| Cellulose acetate with 39.8% acetate | 9.5 mg |
| Macrogol 3350 | 0.5 mg |
| Titanium dioxide | 0.5 mg |

### Quantities Stated Per 100 mg of Antagonist Pellets

Cellulose acetate and macrogol were dissolved in an acetone-water mixture (95:5 parts by weight), titanium dioxide was dispersed in the mixture and the cores were sprayed with the suspension in a fluidised bed unit until the mass of the coated pellets amounted to 110% of the weight of the introduced uncoated pellets.

### Analgesic Pellets

| | |
|---|---|
| 0.5 mm nonpareils (sucrose-maize starch starter pellets, supplied | 50 mg |
| by Werner) | |
| Morphine sulfate pentahydrate | 60 mg |
| Povidone K30 | 30 mg |
| Talcum | 10 mg |

Morphine sulfate and povidone were dissolved in purified water and talcum was dispersed in the solution. The suspension was sprayed onto the nonpareils at 60° C. and dried. The pellets were classified by means of a 1.5 mm and a 0.5 mm screen.

### Coating on Analgesic Pellets

| | |
|---|---|
| Ethylcellulose dispersion (Aquacoat ECD30, FMC Corporation) | 10.0 mg |
| Glycerol monostearate | 2.0 mg |
| Talcum | 2.0 mg |
| Titanium dioxide | 1.0 mg |

Quantities stated per 150 mg of analgesic pellets, weight of ethylcellulose stated as the dry weight obtained from the 30% dispersion of the commercial product.

The ethylcellulose dispersion was mixed 1:0.5 with purified water and the glycerol monostearate was incorporated by stirring for at least two hours. Talcum and titanium dioxide were dispersed in 0.5 parts of water (calculated on the basis of the 1:0.5 mixture of the ethylcellulose dispersion) and mixed with the ethylcellulose dispersion. The analgesic pellets were sprayed with the dispersion in a fluidised bed unit until the mass of the coated pellets amounted to 110% of the weight of the introduced uncoated pellets.

### Final Formulation in Capsules

55 mg of coated pellets containing antagonist and 165 mg of coated analgesic pellets per capsule were mixed and packaged in size 2 hard gelatine capsules.

### Example 6

### Oral Osmotic Therapeutic System

### Active Ingredient Layer

| | |
|---|---|
| Morphine sulfate pentahydrate | 125 mg |
| Macrogol 200,000 | 280 mg |
| Povidone (MW_{N} 40,000) | 26 mg |
| Magnesium stearate | 4 mg |

The morphine sulfate and macrogol were dry-mixed in a planetary mixer and then converted into a moist mass by slow addition of a solution of the povidone in 115 mg of ethanol and the mass was then pressed through a 0.8 mm screen. After 24 hours' drying at room temperature in a fume hood, the particles were pressed together with the magnesium stearate through a 1.0 mm screen and mixed in a container mixer.

### Push Layer

| | |
|---|---|
| Methylhydroxypropylcellulose 6 mPa · s | 13 mg |
| Sodium chloride | 80 mg |
| Macrogol 7,000,000 | 166 mg |
| Magnesium stearate | 1 mg |

The sodium chloride, macrogol and half the methylhydroxypropyl-cellulose were dry-mixed for 3 minutes in a fluidised bed granulator and then granulated and dried by spraying on a solution of the second half of the methylhydroxypropylcellulose in 75 mg with introduction of hot air. The granular product was then pressed together with the magnesium stearate through a 2.5 mm screen in a Comil.

### Antagonist Layer

| | |
|---|---|
| Naltrexone hydrochloride | 50 mg |
| Hydrogenated castor oil (Cutina HR) | 60 mg |
| Lactose | 20 mg |

Naltrexone hydrochloride and hydrogenated castor oil were precompressed in a tablet press with a 10 mm precompression punch to form approx. 250 mg compression moldings. The preliminary compression moldings were then comminuted by means of a crusher and a 1.0 mm screen.

### Production of the 3 Layer Tablets

For each tablet, 100 mg of the granular product for the antagonist layer, 260 mg of the push layer and 435 mg of the active ingredient layer were introduced in succession into the die of a suitable tablet press and press-molded to form a 3 layer tablet.

### Coating on Core

| | |
|---|---|
| Cellulose acetate with 39.8% acetate | 38 mg |
| Macrogol 3350 | 2 mg |

The coating constituents were dissolved as a 3.8% solution in an acetone-water mixture (95:5 parts by weight) and sprayed onto the cores. Two 0.75 mm diameter holes were drilled through the coating in order to connect the active ingredient layer with external environment of the system.

### Example 7

### Oral Osmotic Therapeutic System

Production proceeded in a manner similar to Example 6, except that the antagonist layer was of the following composition:

| | |
|---|---|
| Naloxone hydrochloride dihydrate | 60 mg |
| Hydrogenated castor oil (Cutina HR) | 40 mg |
| Lactose | 20 mg |

Naloxone hydrochloride dihydrate, hydrogenated castor oil and lactose were precompressed in a tablet press with a 10 mm precompression punch to form approx. 250 mg compression moldings. The preliminary compression moldings were then comminuted by means of a crusher and a 1.0 mm screen. All the other production steps proceeded as explained in Example 6.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. An abuse-resistant, oral, solid pharmaceutical dosage form comprising at least one active ingredient with potential for abuse and at least one antagonist for this active ingredient spatially separate from the active ingredient, wherein said at least one active ingredient is present in at least one subunit (a), and said at least one antagonist is present in at least one subunit (b), and wherein:
subunits (a) and (b) are both in multiparticulate form, and the multiparticulate forms of subunits (a) and (b) are substantially identically shaped and are not visually distinguishable,
and wherein if the dosage form is correctly administered to an individual, the at least one antagonist is not released in the body of said individual.

2. A dosage form according to claim 1, wherein subunits (a) and (b) are in multiparticulate form and the particles of subunits (a) and (b) are packaged together in capsules or suspended together in a liquid or a gel.

3. A dosage form according to claim 1, wherein said at least one active ingredient is selected from the group consisting of opiates, opioids, stimulants and non-opiate narcotics.

4. A dosage form according to claim 3, wherein said at least one active ingredient is selected from the group consisting of: N-{1-[2-(4-ethyl-5-oxo-2-tetrazolyl-1-yl)ethyl]-4-methoxymethyl-4-piperidyl}propionanilide (Alfentanil), allylprodine, alphaprodine, 2-diethylaminopropiophenone (amfepramone), (±)-α-methylphenethylamine (amphetamine), 2-(α-methylphenethylamino)-2-phenylacetonitrile (amphetaminil), anileridine, apocodeine, benzylmorphine, bezitramide, 17-cyclopropylmethyl-4,5α-epoxy-7α[(S)-1-hydroxy-1,2,2-trimethyl-propyl]-6-methoxy-5,14-endo-ethanomorphinan-3-ol (buprenorphine), butorphanol, (1S,2S)-2-amino-1-phenyl-1-propanol (cathine/D-norpseudoephedrine), clonitazene, (−)-methyl-[3β-benzoyloxy-2β(1αH,5αH)-tropane carboxylate] (cocaine), 4,5α-epoxy-3-methoxy-17-methyl-7-morphinen-6α-ol (codeine), cyclorphan, cyprenorphine, desomorphine, dextromoramide, (+)-(1-benzyl-3-dimethylamino-2-methyl-1-phenylpropyl) propionate (dextropropoxyphene), dezocine, diampromide, diamorphone, 4,5α-epoxy-3-methoxy-17-methyl-6α-morphinanol (dihydrocodeine), 4,5α-epoxy-17-methyl-3,6α-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10α-tetrahydro-6H-benzo[c]chromen-1-ol (dronabinol), eptazocine, ethoheptazine, ethylmethylthiambutene, 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-(α-methylphenethylamino)propionitrile (fenproporex), N-(1-phenethyl-4-piperidyl)propionanilide (fentanyl), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 5-(4-chlorophenyl)-2,5-dihydro-3H-imidazo[2,1-α]isoindol-5-ol (mazindol), N-(3-chloropropyl)-α-methylphenethylamine (mefenorex), meperidine, meptazinol, metazocine, methylmorphine, N,α-dimethylphenethylamine (metamphetamine), (±)-6-dimethylamino-4,4-diphenol-3-heptanone (methadone), methyl[2-phenyl-2-(2-piperidyl)acetate] (methylphenidate), 3,3-diethyl-5-methyl-2,4-piperidinedione (methyprylon), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-[b,d]pyran-9(6αH)-one (nabilone), nalbuphene, narceine, nicomorphine, norlevorphanol, 6-dimethylamino-4,4-diphenyl-3-hexanone (normethadone), normorphine, norpipanone, the exudation from plants belonging to the species Papaver somniferum (opium), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papavereturn, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), α,α-dimethylphenethylamine (phentermine), α-(2-piperidyl)benzhydryl alcohol (pipradrol), 1′-(3-cyano-3,3-diphenylpropyl)[1,4′-bipiperidine]-4′-carboxamide (piritramide), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate}(remifentanil), N-{4-methoxymethyl-1-[2-(2-thienyl)ethyl]-piperidyl}propionanilide (sufentanil), ethyl (2-dimethylamino-1-phenyl-3-cyclohexene-1-carboxylate) (tilidine, cis and trans)), tramadol, (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, esters and ethers of any of the foregoing, and pharmaceutically acceptable salts and solvates of any of the foregoing.

5. A dosage form according to claim 3, wherein said at least one active ingredient comprises a stimulant selected from the group consisting of amphetamine, norpseudoephedrine, methylphenidate, and physiologically acceptable salts and solvates thereof.

6. A dosage form according to claim 3, wherein said at least one antagonist comprises an opiate or opioid antagonist selected from the group consisting of naloxone, naltrexone, nalmefene, nalide, nalmexone, nalorphine, naluphine, and physiologically acceptable salts and solvates thereof.

7. A dosage form according to claim 3, wherein said at least one antagonist comprises a neuroleptic selected from the group consisting of haloperidol, promethazine, fluphenazine, perphenazine, levomepromazine, thioridazine, perazine, chlorpromazine, chlorprothixine, zuclopentixol, flupentixol, prothipendyl, zotepine, benperidol, pip amperone, melperone and bromperidol.

8. A dosage form according to claim 1, wherein said at least one active ingredient is present at least partially in delayed-release form.

9. A dosage form according to claim 1, wherein said dosage form is provided with a coating resistant to gastric juices.
